(19) United States
(12) Patent Application Publication
(10) Pub. No.: US 2009/0136559 A1
(43) Pub. Date: May 28, 2009

(54) Chondrocyte Differentiation from Human Embryonic Stem Cells and Their Use in Tissue Engineering

(76) Inventors: Kyriacos A. Athanasiou, Houston, TX (US); Gwendolyn Hoben, Houston, TX (US); Eugene Koay, Houston, TX (US); Jerry Hu, Houston, TX (US)

Correspondence Address: BAKER BOTTS, LLP, 910 LOUISIANA, HOUSTON, TX, 77002-4995, US

(21) Appl. No.: 12/246,306

(22) Filed: Oct. 6, 2008

Related U.S. Application Data
(63) Continuation-in-part of application No. 11/571,790.
Application No. 11/571,790 is a continuation of application No. PCT/US05/24269, filed on Jul. 8, 2005.
Application No. PCT/US05/24269 is a continuation-in-part of application No. PCT/US2007/066089, filed on Apr. 5, 2007.
Application No. PCT/US2007/066089 is a continuation-in-part of application No. PCT/US2007/066085, filed on Apr. 5, 2007.
Application No. PCT/US2007/066085 is a continuation-in-part of application No. PCT/US2007/066092, filed on Apr. 5, 2007.
(60) Provisional application No. 60/586,862, filed on Jul. 9, 2004.
(60) Provisional application No. 60/789,853, filed on Apr. 5, 2006.

(51) Int. Cl. A61F 2/00; C12N 5/08; A61K 35/12

(52) U.S. Cl. 424/423; 435/366; 435/377; 435/347; 424/93.7

(57) ABSTRACT

Methods for inducing differentiation of human embryonic stem cells into chondrocytes for use in tissue engineering applications are provided. One example of a method is a method for inducing differentiation of human embryonic stem cells into chondrocytes comprising aggregating undifferentiated human embryonic stem cells to form embryoid bodies; and culturing the embryoid bodies in culture medium in the presence of growth factors that induce chondrogenic differentiation of the embryoid bodies.

### CROSS-REFERENCE TO RELATED APPLICATIONS

This application is a continuation-in-part of application Ser. No. 11/571,790 filed Jan. 8, 2007, which claims the benefit of International Application No. PCT/US2005/24269 filed Jul. 8, 2005, which claims the benefit of U.S. Provisional Application Ser. No. 60/586,862 filed on Jul. 9, 2004; and also a continuation-in-part of International Application Nos. PCT/US2007/066089, PCT/US2007/066085, and PCT/US2007/066092 all filed Apr. 5, 2007, and all of which claim the benefit of U.S. Provisional Application Nos. 60/789,851, 60/789,853, and 60/789,855 all filed Apr. 5, 2006, all of which are incorporated herein by reference.

### STATEMENT OF GOVERNMENT INTEREST

This disclosure was developed at least in part using funding from the National Institutes of Health, Grant Number R01 AR47839-2, and the National Science Foundation-Integrative Graduate Education and Research Traineeship Program, Grant Number DGE-0114264. The U.S. government may have certain rights in the invention.

### BACKGROUND

Tissue engineering is an area of intense effort today in the field of biomedical sciences. The development of methods of tissue engineering and replacement is of particular importance in tissues that are unable to heal or repair themselves, such as articular cartilage. Articular cartilage is a unique avascular, aneural and alymphatic load-bearing live tissue, which is supported by the underlying subchondral bone plate. Articular cartilage damage is common and does not normally self-repair. Challenges related to the cellular component of an engineered tissue include cell sourcing, as well as expansion and differentiation. Findings of recent well-designed studies suggest that autologous chondrocyte implantation is the most efficacious technique of repairing symptomatic full-thickness hyaline articular cartilage defects, which engender a demand for cell-based strategies for cartilage repair. Further studies have also attempted to engineer cartilage via the combination of biodegradable or biocompatible scaffolds with differentiated chondrocytes. According to these studies, it is unlikely that a sufficient supply of differentiated chondrocytes will be available for clinical applications.

To overcome the deficiency in the supply of differentiated chondrocytes, alternate sources of cells from tissues other than cartilage have been researched. A number of researchers have investigated various adult tissues including bone marrow, muscle, and adipose tissue as alternative cell sources for cartilage tissue engineering. However, autologous procurement of these tissues has potential limitations. Stem cells represent a valuable source for this purpose.

A progenitor cell, also referred to as a stem cell, is generally considered an undifferentiated cell that can give rise to other types of cells. A progenitor cell has the potential to develop into cells with a number of different phenotypes. Differentiation usually involves the selective expression of a subset of genes, which vary from cell type to cell type, without the loss of chromosomal material. Thus, the lineal descendants of a progenitor cell can differentiate along an appropriate pathway to produce a fully differentiated phenotype. All differentiated cells have, by definition, a progenitor cell type, for example, neuroblasts for neurons and germ cells for gamete cells.

Progenitor cells share the three following general characteristics: (1) the ability to differentiate into specialized cells, i.e., not terminally differentiated, (2) the ability to regenerate a finite number of times, and (3) the ability to relocate and differentiate where needed. Progenitor cells may give rise to one or more lineage-committed cells, some of which are also progenitor cells, that in turn give rise to various types of differentiated cells and tissues. Progenitor cells generally constitute a small percentage of the total number of cells present in the body and vary based on their relative level of commitment to a particular lineage. Because progenitor cells have the ability to produce differentiated cell types, they may be useful, among other things, for replacing the function of aging or failing cells in many tissues and organ systems.

There are three major classes of progenitor cells, based on what they have the potential to become. The earliest cells, from the fertilized egg through the first few division cycles, are totipotent. A totipotent cell has the genetic potential to create every cell of the body, including the placenta and extra-embryonic tissues.

Next come the pluripotent, or multipotent, cells, which can become more than one kind of cell, but do not have the potential to become all cell types. A pluripotent cell (i.e., an embryonic progenitor cell) has the potential to create every cell of the body, but not the necessary placenta and extra-embryonic tissues required to form a human being. Pluripotent cells can be isolated from embryos and the germ line cells of fetuses. A multipotent cell, or a multipotent adult progenitor cell (“MAPC”), can give rise to a limited number of other particular types of cells. Multipotent cells are found in both developing fetuses and fully developed human beings and have been observed to develop into a variety of cell types such as cardiomyocytes, hepatocytes, and epithelial cells. For example, hematopoietic cells (blood cells) in the bone marrow are multipotent and give rise to the various types of blood cells, including red blood cells, white blood cells, and platelets. Unlike pluripotent cells, multipotent cells are often present in a fully developed human being. But multipotent cells may only be present in minute quantities, and their numbers can decrease with age. Multipotent cells from a specific patient may take time to mature in culture in order to produce adequate amounts for treatment.

And finally there are unipotent cell types, such as the muscle-cell progenitors. These still have the quality of regenerating, but may be more differentiated or committed to a certain cell type.

### DRAWINGS

Some specific example embodiments of the disclosure may be understood by referring, in part, to the following description and the accompanying drawings.

FIG. 1 is a schematic diagram describing one example of a method of using human embryonic stem cells to tissue engineer articular cartilage using a process that does not involve the use of exogenous scaffolds.

FIG. 2 is an image of embryoid bodies after four weeks of culture, according to one embodiment of the present disclosure

FIG. 3 is a photomicrograph image of embryoid body morphology after analysis with A) immunohistochemistry for collagen type II, and B) alcian blue staining for glycosaminoglycans.

FIG. 4 is an image of the gross morphology of constructs after 2 weeks of tissue engineering. FIG. 4A shows a construct with a thickness of approximately 1 mm. FIG. 4B shows a construct with a diameter of 3 mm. Distance between each bar is 1 mm.

FIG. 5 is a photomicrograph image of constructs made with A) 0% serum and B) 20% serum. Shown are collagen type II (left column) and glycosaminoglycan (right column) stained constructs.

FIG. 6 shows chondrogenic differentiation of BG01V and H9. Embryoid Bodies were treated with one of two differentiation regimens. Collagen type II and Alcian blue staining were observed in both cells lines with all serum levels tested. Staining at t=4 wks is shown for representative embryoid bodies. FIG. 6A shows results for 20% FBS BG01V Embryoid Bodies, and FIG. 6B shows results for 0% FBS H9 Embryoid Bodies.

FIG. 7 shows representative constructs for each cell line. A) Self-assembled construct (t=2 wks of self-assembly) made from chondrogenically-differentiated BG01V Embryoid Bodies. For 2 wks of self-assembly, constructs received TGF-β1+IGF-I. This particular construct received no serum. Constructs that received 1% and 20% serum looked similar to this construct. B) Self-assembled construct (t=4 wks of self-assembly) made from chondrogenically-differentiated H9 cells. Pictured is a construct that received 20% FBS and TGF-β1+IGF-I throughout self-assembly. Constructs that received 0% and 1% serum looked similar to this construct. The markings are 1 mm apart.

FIG. 8 shows the expression of collagen type II in self-assembled constructs. After 2 wks of self-assembly, these representative constructs exhibit collagen type II, which was seen after the differentiation phase of 4 wks, suggesting that the chondrocytic phenotype is maintained. A) Shown is collagen type II staining for the sample pictured in FIG. 7A (0% FBS). B) This construct received 20% FBS but the same differentiation agents and growth factors as (A). Both were BG01V constructs.

FIG. 9 shows analysis for chondrogenic differentiation of hESCs at t=4 wks FIG. 9A shows collagens type I and II detected in all three differentiation conditions with immunohistochemistry at t=4 wks (10×). The EBs in all groups appeared highly hydrated and cellular with a loosely organized ECM. Due to this, obtaining good frozen sections for these structures was challenging. Calcified tissue (i.e., bone), muscle, adipose were not detected (data not shown). FIG. 9B shows that SOX-9 transcription factor was detected in all three differentiation regimens at t=4 wks (green). The blue fluorescence is a Hoechst stain for the nucleus. While CM and D1 cells were approximately the same size and had a similar rounded shape as the positive control of native articular chondrocytes (bottom row, left), D2 cells were larger and appeared fibroblastic. The negative control of MEFs (bottom row, right) did not stain for SOX-9. The white bar is 10 μm (40×).

FIG. 10 shows gross morphology and histology of self-assembled constructs at t=8 wks. FIG. 10A shows that dissociated cell (DC) constructs appeared more uniform than embryoid body (EB) constructs. The DC group also held their shape when manipulated, while the EB group did not. D1 constructs were generally smaller than constructs from the other two groups, as shown in the pictures and the morphological measurements. EB constructs were engineered larger (5-mm molds vs 3-mm molds for DC constructs) because the EBs at t=4 wks were too large for the 3-mm wells. FIG. 10B shows that collagens I and II (top two rows) were detected in CM and D2 groups with immunohistochemistry at t=8 wks, regardless of self-assembly mode (EB or DC) (10×). D1 constructs had collagen type II but did not demonstrate much collagen type I staining. Intense picrosirius red and spotty Alcian blue stains (4×) are shown in the bottom row for each differentiation condition. Calcified tissues (i.e., bone), muscle, and adipose were not detected at t=8 wks (data not shown).

FIG. 11 shows biochemical analysis of total collagen and sulfated GAGs at t=8 wks. FIG. 11A illustrates that self-assembly with DCs caused an increase in total collagen content compared to self-assembly with EBs (p=0.002). Significant differences were also detected due to differentiation agent, with CM and D2 constructs being higher than D1 constructs (p=0.0007). Note: The convention used to show statistically different results are upper or lower case letters (one set for each experimental factor). Groups not connected by the same letter are significantly different (p<0.05). FIG. 11B shows that sulfated GAG content was higher in DC constructs compared to EB constructs (p=0.038). Differentiation condition was not a significant factor for GAG production.

FIG. 12 shows ELISAs for collagens I and II. FIG. 12A illustrates that the picogreen results from the ELISA digest showed that CM constructs had higher cell numbers than D1 constructs at t=8 wks. Particularly notable is the fact that CM dissociated cell (DC) constructs had almost twice as many cells as the other two DC groups. All constructs were initially seeded with the same amount of cells. Additionally, D1 embryoid body (EB) constructs exhibited lower cell numbers than the other EB constructs. These results generally mirror the gross morphology of the constructs. FIG. 12B shows that collagen type I per cell was undetectable in D1 constructs, while CM and D2 constructs exhibited relatively high amounts of collagen type I per cell. Overall, CM constructs had higher collagen type I content (p<0.0001). Also, DC constructs had more collagen type I per cell than EB constructs (p<0.0001). FIG. 12C shows that collagen type II per cell demonstrated differences between EB and DC constructs (p=0.008). CM constructs had more collagen type II per cell than D2 constructs (p=0.001).

FIG. 13 shows compressive properties of the constructs at t=8 wks. Dissociated cell (DC) constructs had a higher instantaneous modulus than embryoid body (EB) constructs (p=0.005). Differentiation condition had no effect.

FIG. 14 shows the tensile properties of dissociated cell (DC) constructs at t=8 wks. FIG. 14A shows that DC constructs had enough mechanical integrity to be tested under tension, while embryoid body (EB) constructs did not have this degree of mechanical integrity and could not be tested. In terms of both tensile modulus and ultimate tensile strength, D2 constructs were significantly higher than CM and D1 constructs. Also notable was the fact that the values for these properties were on the order of megapascals. FIG. 14B shows that collagen alignment (demonstrated by picrosirius red and polarized light) in the specimens along the axis of tensile testing (double headed arrow) was seen best in the D2 group, while the CM and D1 specimens demonstrated no preferred direction (top row). Pictured on the top row are one-half of the tensile specimens, with the broken end (where failure occurred) being on the left of each picture (white arrow, 10×). Analyzing the untested whole constructs (bottom row) also demonstrated a higher degree of collagen alignment in D2 constructs compared to the other groups (10×).

The patent or application file contains at least one drawing executed in color. Copies of this patent or patent application publication with color drawing(s) will be provided by the Office upon request and payment of the necessary fee.

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as illustrated, in part, by the appended claims.

### DESCRIPTION

The present disclosure is generally in the field of improved methods for tissue engineering. More particularly, the present disclosure relates to methods for inducing differentiation human embryonic stem cells to serve as a source of chondrocytes and associated methods of use in forming tissue engineered constructs.

The methods of the present disclosure generally comprise aggregating undifferentiated human embryonic stem cells to form embryoid bodies; and culturing the embryoid bodies in culture medium in the presence of growth factors that induce chondrogenic differentiation of the embryoid bodies.

In certain other embodiments, the methods of the present disclosure comprise aggregating undifferentiated human embryonic stem cells to form embryoid bodies; culturing the embryoid bodies in culture medium in the presence of growth factors that induce chondrogenic differentiation of the embryoid bodies; sedimenting the differentiated embryoid bodies onto a hydrogel coated culture vessel; and allowing the differentiated embryoid bodies to self-assemble to form a construct. The term “human embryonic stem cell” is defined herein to include cells that are self-replicating or can divide and to form cells indistinguishable from the original, derived from human embryos or human fetal tissue, and are known to develop into cells and tissues of the three primary germ layers, the ectoderm, mesoderm, and endoderm. Although human embryonic stem cells may be derived from embryos or fetal tissue, such stem cells are not themselves embryos. The term “embryoid bodies” is defined herein to include any cluster or aggregate of human embryonic stem cells. The term “chondrogenic differentiation” is defined herein to include any process that would result in cells that produce glycosaminoglycans and collagen type II.

The term “construct” or “tissue engineered construct” as used herein refers to a three-dimensional mass having length, width, and thickness, and which comprises living mammalian tissue produced in vitro. As used herein, “self-assemble” or “self-assembly” as used herein refers to a process in which specific local interactions and constraints between a set of components cause the components to autonomously assemble, without external assistance, into the final desired structure through exploration of alternative configurations.

Among other things, the methods of the present disclosure may be used to produce human cartilage constructs. Another advantage of the methods of the present disclosure is that human embryonic stem cells can be easily expanded in culture, and human embryonic stem cells possess the ability to maintain their phenotype stably in culture theoretically over limitless numbers of passages (an immortal cell line), while native chondrocytes and other stem cells will lose their phenotype when expanded over just a few passages. In addition to their expansion capability, the pluripotency of human embryonic stem cells makes them attractive for various regenerative medicine approaches, including cartilage tissue engineering. These features are especially attractive for cartilage tissue engineering, where scarcity of chondrocytes is considered a major impediment. Establishing human embryonic stem cells for this purpose requires a protocol for chondrogenic differentiation and a method to harness the cells' synthetic potential. The methods of the present disclosure may provide for the specific formation of cartilage, at least until 6 weeks of total culture, which is apparent due to the lack of other tissues in our engineered constructs. In certain embodiments, the methods of the present disclosure do not involve the use of fetal bovine serum, which is an animal product. The ability to produce constructs without the use of fetal bovine serum is a milestone that may ease the translation of the present disclosure to therapeutic applications.

The present disclosure also provides for a system for studying tissue engineering with human embryonic stem cells that can discern functional differences between engineered cartilages made from chondrogenically-differentiated human embryonic stem cells that were exposed to distinct differentiation conditions.

The modular design of this tissue engineering methodology accommodates perturbations to each of the key components during each phase to study how human embryonic stem cells differentiate and how these differentiated cells can be used to engineer cartilage. With this system, a number of investigations into the effects of different seeding densities, different growth environments, and other biochemical and biomechanical differentiation agents can be imagined. The developed methodology can also be used as a model system for fundamental research.

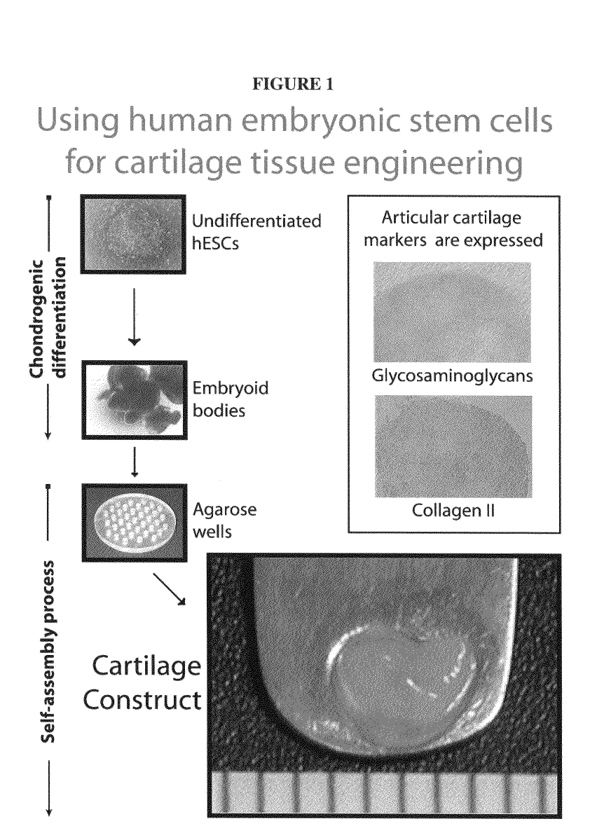

Referring initially to FIG. 1, a schematic diagram of the process of utilizing undifferentiated human embryonic stem cells to form tissue engineered constructs, the methods of the present disclosure generally comprise aggregating undifferentiated human embryonic stem cells to form embryoid bodies, culturing the embryoid bodies in culture medium in the presence of growth factors that induce chondrogenic differentiation of the embryoid bodies, sedimenting the differentiated embryoid bodies onto a hydrogel coated culture vessel, and allowing the differentiated embryoid bodies to self-assemble to form a construct.

Source of Undifferentiated Human Embryonic Stem Cell

The human embryonic stem cells suitable for use in conjunction with the methods of the present disclosure can be obtained from a variety of sources. For example, two NIH-approved human embryonic stem cell lines, BG01V and H9 may be used in conjunction with the methods of the present disclosure. The human embryonic stem cells may be cultured according to standard embryonic cell culture protocols available to those of ordinary skill in the art. For example, undifferentiated cells may be derived from induced pluripotent stem cells.

Alternatively, the cells may be obtained from an embryonic stem cell bank or from the process of somatic cell nuclear transfer. An embryonic stem cell bank containing 150 human embryonic stem cell lines could be used for KLA (antigen) matching a human embryonic stem cell line to about 85% of all possible recipients (published in Lancet, December 2005). The principles described herein could be applied to any of these human embryonic stem cell lines to produce tissue engineered constructs with minimal possibility of immune rejection.

Somatic cell nuclear transfer would involve the creation of a patient-specific human embryonic stem cell line by transferring genetic material from one of the patient's adult cells (i.e., a skin cell) to an unfertilized human ovum. After 5 days in culture, human embryonic stem cells can be derived from the inner cell mass and treated with the methods described herein to obtain patient-specific construct.

Culture Medium

One of ordinary skill in the art, with the benefit of this disclosure, will recognize that suitable culture medium should be used in conjunction with the methods of the present disclosure such that human embryonic stem cells may proliferate and preferably such that stem cells may aggregate to form embryoid bodies, and be induced to differentiate. In certain embodiments, the medium used may comprise fetal bovine serum. The fetal bovine serum may be present in the range of about 1% to about 20% of culture medium. In certain embodiments, the culture media may be substantially free of fetal bovine serum. The ability to produce constructs without the use of fetal bovine serum is an advantage of the methods of the present disclosure that may ease the translation of the present disclosure to therapeutic applications. One example of suitable medium for use in conjunction with the methods of the present disclosure is medium comprising high glucose Dulbecco's Modified Eagle Medium (DMEM), 10⁻⁷ M dexamethasone, 50 μg/ml ascorbic acid, 40 μg/ml L-proline, 100 μg/ml sodium pyruvate, 1% FBS, and ITS+Premix (6.25 ng/ml insulin, 6.25 mg/ml transferrin, 6.25 ng/ml selenious acid, 1.25 mg/ml bovine serum albumin, and 5.35 mg/ml linoleic acid).

Another example of suitable medium for use in conjunction with the methods of the present disclosure is medium comprising DMEM with 4.5 g/L-glucose and L-glutamine, 0.1 μM dexamethasone, 50 μg/ml ascorbate-2-phosphate, 40 μg/ml proline, 100 μg/ml sodium pyruvate, 1% fungizone, 1% Penicillin/Streptomycin, and 1×ITS+Premix (6.25 μg/ml insulin, 6.25 μg/ml transferrin, 6.25 ng/ml selenious acid, 1.25 mg/ml BSA, and 5.35 mg/ml linoleic acid)

Chondrogenic Differentiation of Undifferentiated Embryoid Bodies

The human embryonic stem cells used in conjunction with the methods of the present disclosure may be aggregated to form embryoid bodies. The embryoid bodies may be differentiated using culture medium in the presence growth factors that induce chondrogenic differentiation. A variety of growth factors can be used in conjunction with the methods of the present disclosure. Suitable examples of growth factors include, but are not limited to, TGF-β1, IGF-I, BMP-2, BMP-4 and TGF-β3.

In certain embodiments, the chondrogenic potential of human embryonic stem cells can be altered with soluble growth factors. In certain embodiments, TGF-β3 may be administered during the critical early period of embryoid body differentiation when the specification of mesodermal cells into precursors of different lineages may occur. After this initial stage, the combination of TGF-β1 with IGF-I or BMP-2 alone may be administered to the embryoid bodies.

In certain embodiments, the embryoid bodies are cultured in medium supplemented by a combination of TGF-β1 and IGF-I. In certain embodiments, the TGF-β1 is present at a concentration of about 10 ng/mL of culture medium. In certain embodiments, the IGF-I may be present at a concentration of 100 ng/mL of culture medium. The embryoid bodies may be exposed to the combination of TGF-β1 and IGF-I for a period of about four weeks.

In certain embodiments, the embryoid bodies may be induced to differentiate by exposure to TGF-β1, IGF-I, and TGF-β3. The TGF-β3 may be exposed to the embryoid bodies in the culture prior to exposure of the embryoid bodies to TGF-β1 and IGF-I. In certain embodiments, the TGF-β3 is present at a concentration of about 10 ng/mL of culture media and is present in the media for a period of about one week. Following the removal of the TGF-β3 from culture, TGF-β1 and IGF-I may be introduced into the medium at a concentration of about 10 ng/mL of culture media and 100 ng/mL of culture media, respectively, for a period of about four weeks.

In certain other embodiments, only TGF-β3 may present at a concentration of about 10 ng/mL of culture media for a period of about one week followed by exposure of the embryoid bodies to BMP-2 at a concentration of about 10 ng/mL of culture medium for a period of about three weeks.

In certain other embodiments, differentiation may be achieved via co-culturing with somatic cells such as chondrocytes, fibrochondrocytes, and synoviocytes.

Hydrogel Coating of Culture Vessels

The culture vessels may be coated with a hydrogel in conjunction with the methods of present disclosure. In certain embodiments, the bottoms and sides of a culture vessel may be coated with 2% agarose (w/v). While 2% agarose is used in certain embodiments, in other embodiments, the agarose concentration may be in the range of about 0.5% to about 4% (w/v). The use of lower concentrations of agarose offers the advantage of reduced costs; however, at concentrations below about 1% the agarose does not stiffen enough for optimal ease of handling.

As an alternative to agarose, other types of suitable hydrogels may be used (e.g. aliginate). A “hydrogel” is a colloid in which the particles are in the external or dispersion phase and water is in the internal or dispersed phase. Suitable hydrogels are non-toxic to the cells, are non-adhesive, do not induce chondrocyte attachment, allow for the diffusion of nutrients, do not degrade significantly during culture, and are firm enough to be handled.

Sedimentation and Self-Assembly of Embryoid Bodies to Form Tissue Engineered Constructs

The chondrogenically differentiated embryoid bodies may be sedimented on hydrogel coated culture vessels. In certain embodiments, the embryoid bodies may be seeded at a concentration of 1×10⁶ cells per well in 3 mm wells with culture medium. In certain embodiments, the culture medium may be supplemented with TGF-β1 and IGF-I. In certain embodiments, the TGF-β1 is present at a concentration of about 10 ng/mL of culture medium. In certain embodiments, the IGF-I may be present at a concentration of 100 ng/mL of culture medium.

In certain embodiments, the amount of growth factor may be varied to provide for tissue engineered constructs with different ranges of collagen that are more representative of the range of collagen found in native tissues.

In certain embodiments, the embryoid bodies may be chemically dissociated prior to sedimentation on the hydrogel coated culture vessels. In certain embodiments, the embryoid bodies may be enzymatically dissociated during the transition from differentiation to self-assembly. This dissociation provides differentiated embryoid bodies that may then be used to produce the tissue engineered constructs of the present disclosure.

In certain embodiments, the embryoid bodies may be pressurized to 10 MPa at 1 Hz using a sinusoidal waveform function. In other embodiments, the embryoid bodies may be pressurized during self-assembly of the embryoid bodies. In particular embodiments, a loading regimen (e.g. compressive, tensile, shear forces) may be applied to the embryoid bodies during self-assembly based on physiological conditions of the native tissue in vivo. Loading of the embryoid bodies during self-assembly and/or construct development may cause enhanced gene expression and protein expression in the constructs.

In particular embodiments, the constructs may be treated with staurosporine, a protein kinase C inhibitor and actin disrupting agent, during the self-assembly process to reduce synthesis of αSMA, a contractile protein. Reducing αSMA in the constructs via staurosporine treatment may reduce construct contraction and may also upregulate ECM synthesis.

Hydrogel Molds

In certain embodiments, the chondrogenically differentiated embryoid bodies may be sedimented on a hydrogel coated culture vessel, allowed to self-assemble into a tissue engineered construct, and molded into a desired shape. In certain embodiments, the self-assembly of the embryoid bodies into a construct may occur on hydrogel coated culture vessels before the construct is transferred to a shaped hydrogel negative mold for molding the construct into the desired shape.

Alternatively, rather than sedimenting the chondrogenically induced embryoid bodies on a hydrogel coated culture vessel, in certain embodiments, the cells may be sedimented directly onto a shaped hydrogel negative mold. The shaped hydrogel negative mold may comprise agarose. Other non-adhesive hydrogels, e.g. alginate, may be used in conjunction with the methods of the present disclosure. In other embodiments, the hydrogel mold may be a two piece structure comprising, a shaped hydrogel negative mold and a shaped hydrogel positive mold. The shaped hydrogel negative and positive molds may comprise the same non-adhesive hydrogel or may be a comprised of different non-adhesive hydrogels.

In certain embodiments, the chondrogenically differentiation embryoid bodies may be sedimented onto a hydrogel coated culture vessel and allowed to self-assemble into a construct. The construct may be transferred to a shaped hydrogel negative mold. A shaped hydrogel positive mold may be applied to the negative mold to form a mold-construct assembly. The mold-construct assembly may then further be cultured. As used herein, the term “mold-construct assembly” refers to a system comprising a construct or cells within a shaped positive and a shaped negative hydrogel mold.

In certain embodiments, the molds may be shaped from a 3-D scanning of a total joint to result in a mold fashioned in the shape of said joint. In other embodiments, the molds may be shaped from a 3-D scanning of the ear, nose, or other non-articular cartilage to form molds in the shapes of these cartilages. In certain embodiments, the mold may be shaped to be the same size as the final product. In other embodiments, the molds may be shaped to be smaller than the final product. In certain embodiments, the molds may be fashioned to a portion of a joint or cartilage so that it serves as a replacement for only a portion of said joint or cartilage.

Other examples of shaped hydrogel molds and methods of developing scaffoldless tissue engineered constructs that may be useful in conjunction with the methods of the present disclosure may be found in co-pending application entitled “A Shape-Based Approach for scaffoldless Tissue Engineering,” the disclosure of which is incorporated by reference herein.

Analysis of the Constructs

The properties of constructs may be tested using any number of criteria including, but not limited to, morphological, biochemical, and biomechanical properties, which also may be compared to native tissue levels. In this context, morphological examination includes histology using safranin-O and fast green staining for proteoglycan and GAG content, as well as picro-sirius red staining for total collagen, immunohistochemistry for collagens I and II, and confocal and scanning electron microscopies for assessing cell-matrix interactions. Biochemical assessments includes picogreen for quantifying DNA content, DMMB for quantifying GAG content, hydroxyproline assay for quantifying total collagen content, and ELISA for quantifying amounts of specific collagens (I and II), and RT-PCR for analysis of mRNA expression of proteins associated with the extracellular matrix (e.g. collagen and aggrecan).

Constructs also may be evaluated using one or more of incremental tensile stress relaxation incremental compressive stress relaxation, and biphasic creep indentation testing to obtain moduli, strengths, and viscoelastic properties of the constructs. Incremental compressive testing under stress relaxation conditions may be used to measure a construct's compressive strength and stiffness. Incremental tensile stress relaxation testing may be used to measure a construct's tensile strength and stiffness. Additionally, indentation testing under creep conditions may be used to measure a construct's modulus, Poisson's ratio, and permeability.

Without wishing to be bound by theory or mechanism, although both collagen type II and glycosaminoglycans (GAGs) are excellent predictors of biomechanical indices of cartilage regeneration, typically only collagen type II exhibits a positive correlation. Though seemingly this hypothesis is counterintuitive for compressive properties, as GAG content is usually thought to correlate positively with compressive stiffness, our results show that in self-assembled constructs, GAG is negatively correlated with the aggregate modulus (R²=0.99), while collagen type II is positively correlated (R²=1.00).

The constructs of the present disclosure may be assessed morphologically and/or quantitatively. Quantitatively, the constructs of the present disclosure may be evaluated using a functionality index (FI) as described in Eq. 1. The functionality index is an equally weighted analysis of ECM production and biomechanical properties that includes quantitative results corresponding to the constructs' salient compositional characteristics (i.e., amounts of collagen type II and GAG) and biomechanical properties (compressive and tensile moduli and strengths).

$$F I = \frac{1}{4} ( ( 1 - \frac{( {G}_{nat} - {G}_{sac} )}{{G}_{nat}} ) + ( 1 - \frac{( {C}_{nat} - {C}_{sac} )}{{C}_{nat}} ) + \frac{1}{2} ( 1 - \frac{( {E}_{nat}^{T} - {E}_{sac}^{T} )}{{E}_{nat}^{T}} ) + \frac{1}{2} ( 1 - \frac{( {E}_{nat}^{C} - {E}_{sac}^{C} )}{{E}_{nat}^{C}} ) + \frac{1}{2} ( 1 - \frac{( {S}_{nat}^{T} - {S}_{sac}^{T} )}{{S}_{nat}^{T}} ) + \frac{1}{2} ( 1 - \frac{( {S}_{nat}^{C} - {S}_{sac}^{C} )}{{S}_{nat}^{C}} ) ) \qquad Eq . ( 1 )$$

In this equation, G represents the GAG content per wet weight, C represents the collagen type II content per wet weight, E^{T} represents the tensile stiffness modulus, E^{C} represents the compressive stiffness modulus, S^{T} represents the tensile strength, and S^{C} represents the compressive strength. Each term is weighted to give equal contribution to collagen, GAG, tension, and compression properties. The subscripts nat and sac are used to denote native and self-assembled construct values, respectively. The aggregate modulus is not used in Eq. 1, as it is expected to mirror the compressive modulus obtained from incremental compressive stress relaxation. Similarly, the amount of collagen type I is not be used in Eq. 1, as this type of collagen may not appear in a measurable fashion; however, if the amount of collagen type I is non-negligible, FI may be altered accordingly to account for it.

Each term grouped in parentheses in Eq. 1 calculates how close each construct property is with respect to native values, such that scores approaching 1 denote values close to native tissue properties. Equal weight is given to GAG, collagen type II, stiffness (equally weighted between compression and tension), and strength (also equally weighted between compression and tension). This index, FI, will be used to assess the quality of the construct compared to native tissue values, with a lower limit of 0 and an unbounded upper limit, with a value of 1 being a construct possessing properties of native tissue. However, the FI can exceed 1 if optimization results in constructs of properties superior to native tissue.

Methods of Using the Tissue Engineered Constructs

In certain embodiments, applications of the tissue engineered construct include the replacement of tissues, such as cartilaginous tissue, the knee meniscus, joint linings, the temporomandibular joint disc, tendons, or ligaments of mammals.

The constructs may be treated with collagenase, chondroitinase ABC, and BAPN to aid in the integration of the constructs with native, healthy tissue surrounding the desired location of implantation. The integration capacity of a construct with native tissue is crucial to regeneration. A wound is naturally anti-adhesive, but debridement with chondroitinase ABC and/or collagenase removes anti-adhesive GAGs and enhances cell migration by removing dense collagen at the wound edge. BAPN, a lysyl oxidase inhibitor, may cause the accumulations of matrix crosslinkers and may, thus, strengthen the interface between the construct and native tissue at the desired location of implantation.

The tissue engineered constructs may be implanted into a subject and used to treat a subject in need of tissue replacement. In certain embodiments, the constructs may be grown in graded sizes (e.g. small, medium, and large) so as to provide a resource for off-the-shelf tissue replacement. In certain embodiments, the constructs may be formed to be of custom shape and thickness. In other embodiments, the constructs may be devitalized prior to implantation into a subject.

To facilitate a better understanding of the present disclosure, the following examples of specific embodiments are given. In no way should the following examples be read to limit or define the entire scope of the disclosure.

### EXAMPLES

### Chondrogenic Differentiation of Human Embryonic Stem Cells

This study investigated the potential of two NIH-approved human embryonic stem cell (HESC) lines, BG01V and H9, to differentiate into cells that produce collagen type II and GAGs. The cell lines were cultured to passages 20-25 using established protocols. To induce the process of differentiation, embryoid bodies (EBs) were formed by exposing undifferentiated HESC colonies to 0.1% (w/v) dispase. Two differentiation agent regimens were used: TGF-β3 (10 ng/ml) for 1 wk followed by TGF-β1 (10 ng/ml)+IGF-I (100 ng/ml) for 3 wks was used with BG01V cells, and TGF-β1 (10 ng/ml)+IGF-I (100 ng/ml) was used with H9 cells for 4 wks. Controls received neither of these differentiation agent regimens. H9 cells received no serum. The BG01V controls and groups exposed to the differentiation agents were tested at three levels of FBS: 0%, 1%, and 20%. EBs were cultured in non-adherent bacteriological petri dishes, and medium was changed every 48 hrs for the duration of the experiment. The medium was composed of DMEM with 4.5 g/L-glucose and L-glutamine supplemented with 0.1 μM dexamethasone, 50 μg/ml ascorbic acid, 40 μg/ml proline, 100 μg/ml sodium pyruvate, and 1×ITS+Premix.

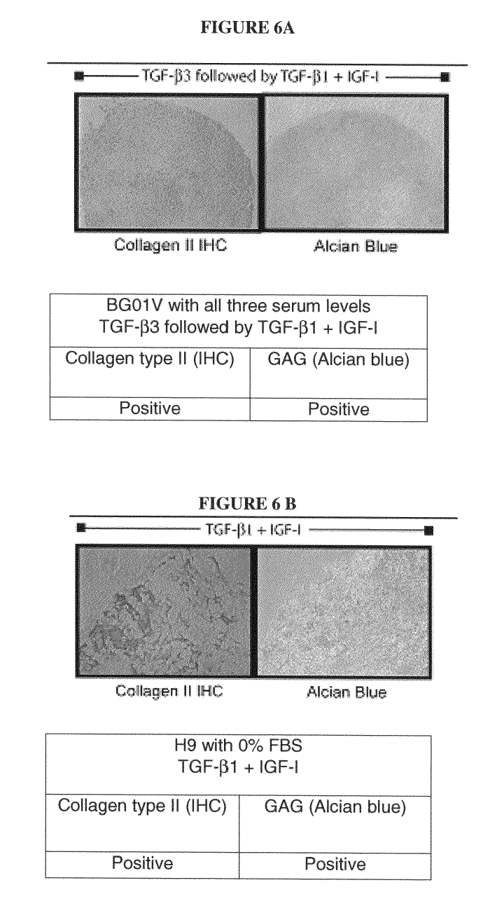

After 4 wks, EBs were cryosectioned at 12 μm, and Alcian blue staining for GAGs and immunohistochemistry for collagen type II were positive with both differentiation agent regimens with all the serum levels tested (FIG. 6). Controls also showed staining for GAGs and collagen type II (data not shown), but the staining was not as consistent as seen with treatment groups. Encouragingly, staining for other tissues was negative, 1) including Oil Red O for adipose tissue, 2) von Kossa for bone, and 3) Masson's Trichrome for muscle (data not shown). In summary, within 4 wks, the results demonstrate that the differentiation agent regimens were able to induce the expression of GAGs and collagen type II in both HESC lines.

Self-Assembly of Chondrogenically-Differentiated hESCs.

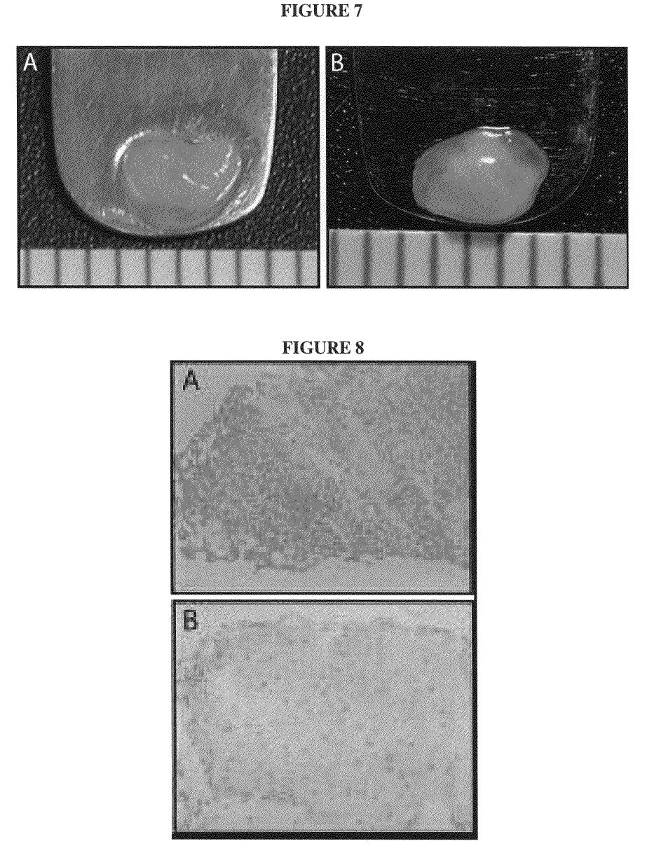

Self-assembly of the BG01V and H9 EBs was initiated by placing enough EBs to cover the bottom of agarose wells (approximately 3×10⁵ cells). Media components were the same as those used for chondrogenic differentiation. This preliminary study used the combination of TGF-β1 (10 ng/ml)+IGF-I (100 ng/ml) for both cell lines, and the serum level used in the differentiation phase stayed the same in the self-assembly phase (0%, 1%, and 20% FBS). The media and growth factors were changed every 48 hrs. After 4 days, the constructs were transferred to 12-well agarose coated plates so that they could grow without confinement. After 2 wks in self-assembly, the BG01V constructs were easily handled and relatively uniform, as shown in FIG. 7A. The H9 constructs were cultured to 4 wks, and appeared similar to the BG01V samples (FIG. 7B). At these time points (t=2 wks for BG01V and t=4 wks for H9), the constructs were cryosectioned at 12 μm and stained using Alcian blue for GAG (data not shown) and immunohistochemistry for collagen type II (FIG. 8). Again, staining was negative for other mesodermal tissues, indicating robust chondrogenic differentiation. The self-assembled constructs were then tested under biphasic creep indentation conditions, yielding compressive modulus values in the same range as those obtained for self-assembled constructs using articular chondrocytes at their respective time points. It was remarkable to note that, using hESCs, tissue engineered constructs of cartilage-like characteristics could be produced with the self-assembly process. Specifically, this study shows that constructs of 1.5 mm thickness and 3 mm dia., with appropriate chondrocytic markers, can be formed using two different hESC lines.

Morphological Assessment of the Embryoid Bodies.

Undifferentiated human embryonic stem cells were incubated with 0.1% (w/v) dispase (Gibco) at 37° C. and 5% CO₂ for 15-30 min, removing colonies intact. The colonies were pelleted and resuspended in medium, consisting of Dulbecco's Modified Eagle Medium (DMEM) with 4.5 g/L-glucose and L-glutamine supplemented with 10⁻⁷ M dexamethasone, 50 μg/ml ascorbic acid, 40 μg/ml proline, 100 μg/ml sodium pyruvate, and 50 mg/ml ITS+Premix (6.25 μg/ml insulin, 6.25 μg/ml transferrin, 6.25 ng/ml selenious acid, 1.25 mg/ml BSA, and 5.35 mg/ml linoleic acid). Additionally, the differentiation was performed at three levels of fetal bovine serum (FBS): 0%, 1%, and 20%. The colonies were placed in 100 mm bacteriological petri dishes (VWR) and formed cell aggregates called embryoid bodies. For directed differentiation, two differentiation regimens were used: 1) Transforming growth factor (TGF)-β1 (10 ng/ml) with Insulin-like growth factor (IGF)-I (100 ng/ml) for 4 wks, and 2) TGF-β3 (10 ng/ml) for 1 wk followed by TGF-β1 (10 ng/ml) with IGF-I (100 ng/ml) for 3 wks. The medium and differentiation agents were replaced together every 48 hours.

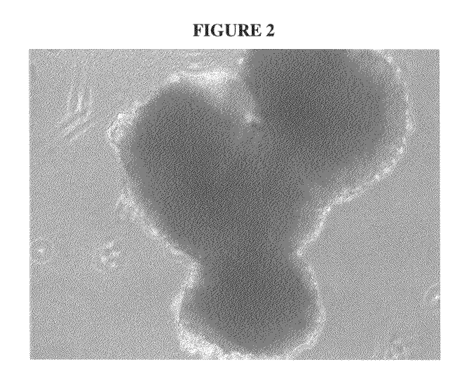

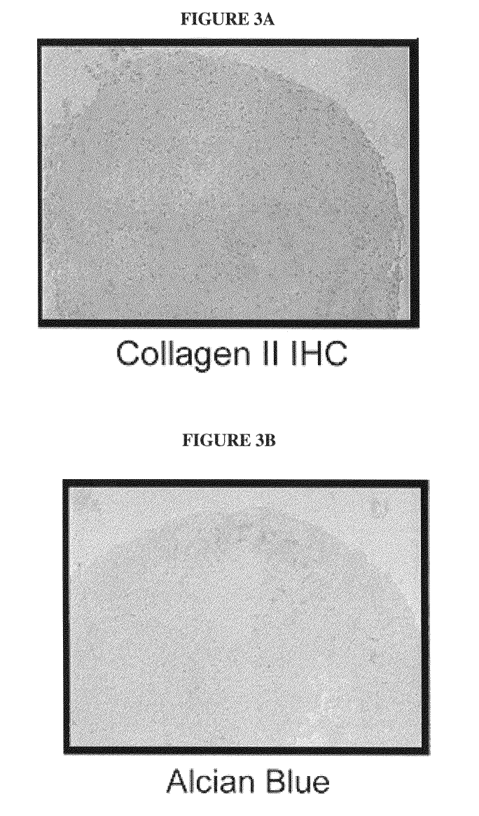

The embryoid bodies (see FIG. 2) were analyzed four weeks after seeding for the articular cartilage specific extracellular matrix proteins glycosaminoglycans and collagen type II using an Alcian blue stain and immunohistochemistry, respectively. Stains for unwanted differentiation in the form of bone (von Kossa), muscle (Masson's Trichrome), and adipose (Oil Red O) were also performed on the constructs. Immunohistochemistry showed production of collagen type II, and histology at this time point demonstrated the presence of abundant glycosaminoglycans for all three levels of FBS (FIG. 3). Other mesodermal tissues were not detected by histology, including bone, muscle, and adipose four weeks after seeding.

Morphological Assessment of the Tissue Engineered Constructs.

Undifferentiated human embryonic stem cells were incubated with 0.1% (w/v) dispase (Gibco) at 37° C. and 5% CO₂ for 15-30 min, removing colonies intact. The colonies were pelleted and resuspended in medium, consisting of Dulbecco's Modified Eagle Medium (DMEM) with 4.5 g/L-glucose and L-glutamine supplemented with 10⁻⁷ M dexamethasone, 50 μg/ml ascorbic acid, 40 μg/ml proline, 100 μg/ml sodium pyruvate, and 50 mg/ml ITS+Premix (6.25 μg/ml insulin, 6.25 μg/ml transferrin, 6.25 ng/ml selenious acid, 1.25 mg/ml BSA, and 5.35 mg/ml linoleic acid). Additionally, the differentiation was performed at three levels of fetal bovine serum (FBS): 0%, 1%, and 20%. The colonies were placed in 100 mm bacteriological petri dishes (VWR) and formed cell aggregates called embryoid bodies. For directed differentiation, two differentiation regimens were used: 1) Transforming growth factor (TGF)-β1 (10 ng/ml) with Insulin-like growth factor (IGF)-I (100 ng/ml) for 4 wks, and 2) TGF-β3 (10 ng/ml) for 1 wk followed by TGF-β1 (10 ng/ml) with IGF-I (100 ng/ml) for 3 wks. The medium and differentiation agents were replaced together every 48 hours.

The bottoms and sides of 96-well plates were coated with 100 μl 2% agarose (w/v), and the plates were shaken vigorously to remove excess agarose. The surface area at the bottom of the well in a 96-well plate is 0.2 cm². Chilled plates were then rinsed with culture medium before the introduction of cells.

After 4 weeks of chondrogenic differentiation, embryoid bodies were placed into hydrogel-coated wells at 1×10⁶ cells per well with 500 μl of culture medium. The medium had the same composition as used during chondrogenic differentiation. The growth factors TGF-β1 (10 ng/ml) with IGF-I (100 ng/ml) were used to culture these constructs.

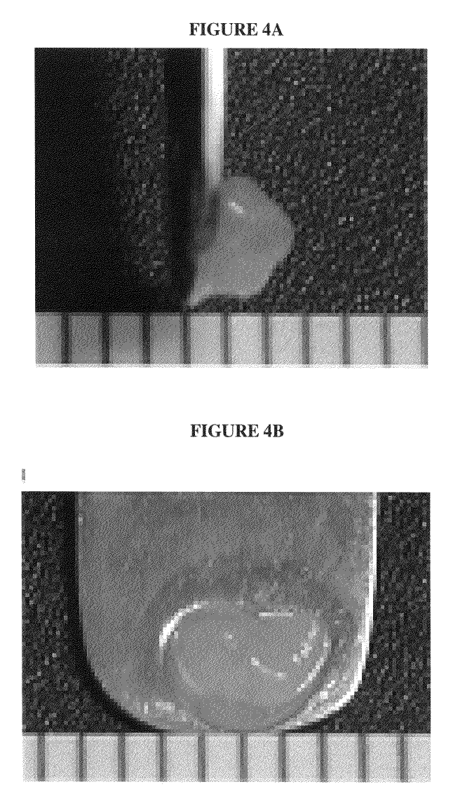

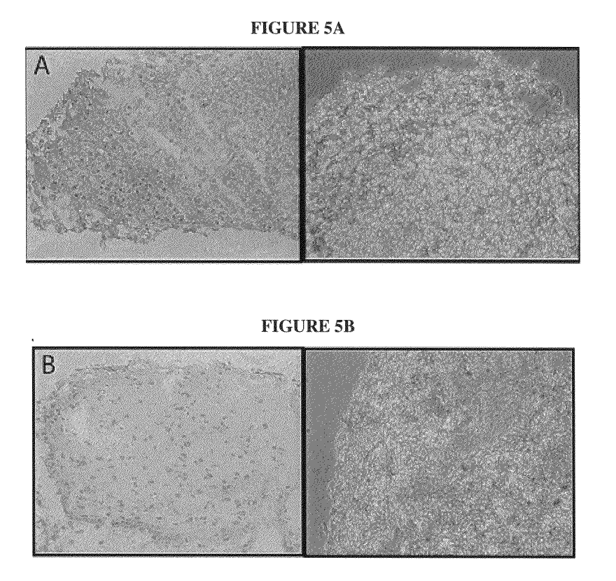

After two weeks of culture on the hydrogel coated tissue culture wells (6 weeks after initial seeding), the developing constructs were analyzed for the articular cartilage specific extracellular matrix proteins glycosaminoglycans and collagen type II using an Alcian blue stain and immunohistochemistry, respectively. Stains for unwanted differentiation in the form of bone (von Kossa), muscle (Masson's Trichrome), and adipose (Oil Red O) were also performed on the constructs. At this time point, the embryoid body constructs were 3 mm in diameter and 1 mm thick (FIG. 4). Glycosaminoglycans and collagen type II are expressed in these constructs at all three levels of FBS (FIG. 5). Other mesodermal tissues were not detected by histology, including bone, muscle, and adipose at this time point.

Determination of the Aggregate Modulus of the Constructs.

After two weeks of culture (6 weeks after initial seeding) on the hydrogel coated wells, the aggregate modulus of the developing constructs was analyzed using prior art techniques. “Aggregate modulus” is a conventional measurement used in characterizing cartilage. Mechanical testing of the representative aggregate or construct yielded a modulus of 6 kPa at 6 weeks after seeding.

Expansion of Human Embryonic Stem Cells.

The NIH-approved HESC line BG01V (American Type Culture Collection, Manassas, Va., http://www.atcc.org) was cultured according to standard protocols. Briefly, a feeder layer of gamma-irradiated CF-1 (Charles River Laboratories, Wilmington, Mass., http://www.criver.com) mouse embryonic fibroblasts (MEFs) at a density of 5×10⁵ MEFs per well of a Nunc 6-well dish (Fisher Scientific, Hampton, N.H., http://www.fishersci.com) was used in the expansion of the hESCs. Frozen hESCs at passage 16 (p16) were thawed according to standard protocol and sub-cultured. A growth medium comprising DMEM/F-12 (Gibco, Gaithersburg, Md., http://www.invitrogen.com), ES-qualified FBS (ATCC), L-glutamine (Gibco), knock out serum replacer (Gibco), and nonessential amino acids (NEAA, Gibco) was used. The hESCs were passaged with collagenase IV (Gibco) every 4-5 days, and cells were utilized for the experiment at p21.

Embryoid Body Formation, Differentiation Conditions, and Analysis.

Dispase solution (0.1% w/v in DMEM/F-12) was applied for 10-15 min to colonies of undifferentiated hESCs in monolayer when the colonies reached 70-80% confluence. This enzymatic treatment predominantly lifts the hESC colonies from the culture dish, leaving MEFs behind and forming embryoid bodies (EBs) from the HESC colonies as described in Zhang S C, Wernig M, Duncan I D, et al. In vitro differentiation of transplantable neural precursors from human embryonic stem cells. Nature Biotech 2001; 19:1129-1133. After two washes and centrifugations with DMEM/F-12, the EBs were suspended in a chondrogenic medium (CM) comprising high-glucose DMEM (Gibco), 10⁻⁷ M dexamethasone, ITS+Premix (6.25 ng/ml insulin, 6.25 mg transferrin, 6.25 ng/ml selenious acid, 1.25 mg/ml bovine serum albumin, and 5.35 mg/ml linoleic acid; Collaborative Biomedical, San Jose, Calif., http://www.bdbiosciences.com), 40 μg/ml L-proline, 50 μg/ml ascorbic acid, 100 μg/ml sodium pyruvate, and 1% FBS (Gemini Bio-Products, West Sacramento, Calif., http://www.gembio.com). The EBs were distributed into bacteriological petri dishes (Fisher) by placing EBs from two 6-well culture plates into each petri dish and using 18 ml of medium per dish. Three differentiation conditions were applied to the EBs in this experiment: 1) CM alone for 28 days (designated CM), (2) CM with TGF-β3 (10 ng/ml) for 7 days followed by the combination of TGF-β1 (10 ng/ml) and IGF-I (100 ng/ml) for 21 days (designated Differentiation Condition 1 (D1)), and (3) CM with TGF-β3 (10 ng/ml) for 7 days followed by BMP-2 (10 ng/ml) for 21 days (designated Differentiation Condition 2 (D2)). For the entire experiment, medium, and, when applicable, growth factors were completely changed every 48 hrs. EBs were used for self-assembly or for histological analysis at t=4 wks.

EBs were also cryo-sectioned and stained for collagens using picrosirius red, GAGs using Alcian blue, and collagen type I and collagen type II using immunohistochemistry (IHC), as previously described in Hu J C and Athanasiou K A. A self-assembling process in articular cartilage tissue engineering. Tissue Eng 2006; 12:969-979. Other stains for mesodermal tissue markers were used to detect unwanted differentiation. These included von Kossa (calcified tissues such as bone), Masson's trichrome (muscle), and Oil red O (adipose). Standard protocols were followed for each of these stains.

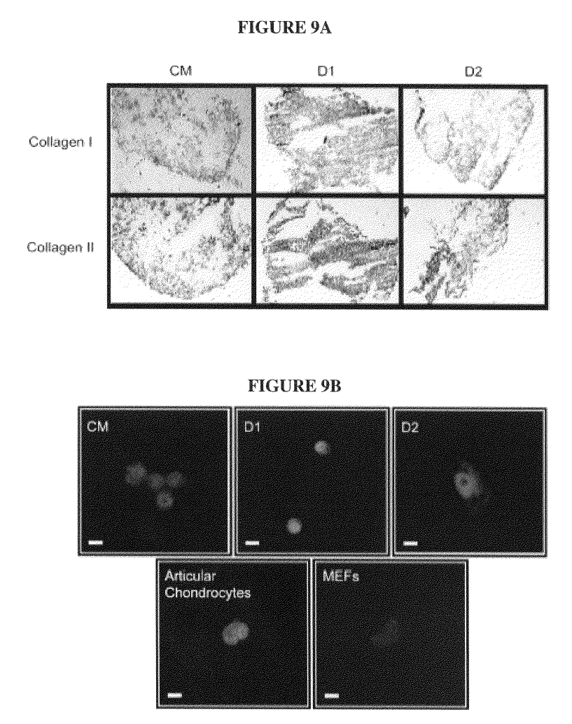

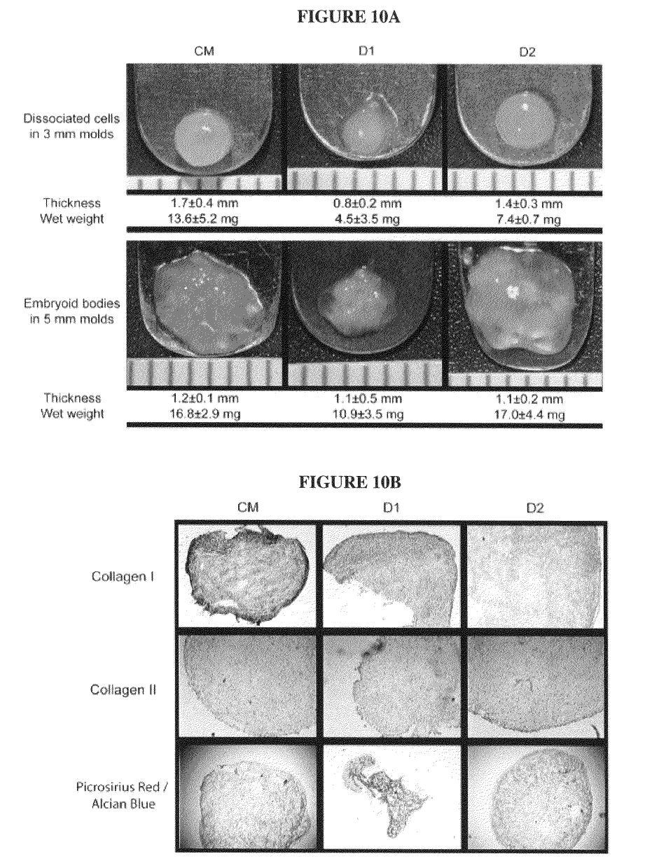

During the 4 wks of differentiation in EB form, EBs noticeably grew in size with the CM (chondrogenic medium without growth factors) and D2 (CM with additives of TGF-β3 followed by BMP-2) groups, while D1 (CM with additives of TGF-β3 followed by TGF-β1 and IGF-I) EBs did not appear to change in size. The morphology and histology of the EBs at t=4 wks is shown in FIG. 9A. The collagen type I and collagen type II IHC illustrate that the cartilaginous matrix in the EBs was loosely connected and unorganized, with all three differentiation conditions exhibiting collagen type I most prominently. Alcian blue staining for all groups at this time point was minimal (data not shown). Dissociation of the EBs with trypsin resulted in a cell suspension, though some cells were still connected with extracellular matrix (ECM) after the 1-hr digestion. Most of the cell suspension was used to make constructs, with at least 8 DC constructs being self-assembled from each differentiation regimen. Similarly, at least 8 EB constructs were self-assembled from each group.

At t=4 wks, a small number of EBs from each differentiation condition were collected for analysis. For visualization of Sox-9, some of the cells obtained from the trypsin digestion at 4 wks of differentiation were plated at a density of 4.0×10⁵ per ml onto a glass slide and allowed to attach overnight. The cells were then fixed with 3.7% paraformaldehyde for 20 min, incubated with Triton-X 100 for 20 min at room temperature, blocked with 3% BSA for 30 min, incubated with Sox-9 primary antibody (Anaspec, Inc., San Jose, Calif.) for 2 hrs, and then incubated with Alexa Fluor® 546 conjugated goat anti-rabbit IgG, secondary antibody (Invitrogen, Carlsbad, Calif., http://www.invitrogen.com) for 1 hr. PBS washes were performed between each of these steps.

A small portion of the cell suspension was used to analyze Sox-9 expression and cell morphology (FIG. 9B). While the cells generated from each differentiation regimen at t=4 wks exhibited Sox-9 protein expression, they exhibited distinct cell morphologies. CM and D1 cells were rounded and approximately the same size as native articular chondrocytes. D2 cells appeared larger and fibroblastic. Histological analyses for calcified tissue (von Kossa), muscle (Masson's trichrome), and adipose (Oil red O) were negative at this time point (data not shown).

### Example 8

### Self-Assembly of Chondrogenically-Differentiated hESCs and Analysis

After 28 days of differentiation (t=4 wks), EBs in each of the three differentiation groups were separated into two equal subgroups. One subgroup of EBs from each differentiation condition was digested in trypsin-EDTA (Gibco) for 1 hr. Cells from each digest were counted with a hemocytometer, washed with DMEM containing 1% FBS, centrifuged at 200×g, and resuspended at a concentration of 5.0×10⁵ cells per 20 μl in CM. Constructs were made by seeding the dissociated cell (DC) suspension into 3 mm wells of 2% agarose (5.0×10⁵ cells per well).

The other subgroup comprised the undigested EBs, which were centrifuged at 200×g and resuspended in 4 ml CM. EBs were seeded into 5 mm wells of 2% agarose using an equivalent of 1×10⁶ cells per construct (based on the hemocytometer count). The two self-assembly modes (EB and DC) were carried out over the ensuing 4 wks, culturing all constructs made from the three differentiation conditions in CM without any exogenous growth factors or stimulation.

At the t=8 wks time point (after 4 wks of self-assembly), each construct was measured for wet weight after carefully blotting excess water. Diameter and thickness measurements were made using digital calipers with an accuracy of 0.01 mm (Mitutoyo, Aurora, Ill., http://www.mitutoyo.com). Constructs were either used for histology, biochemical assays, or biomechanical testing. Histological assessments for self-assembled constructs were exactly the same as that for the EBs (above), except Sox-9 was not assessed at this time point. Additionally, picrosirius red samples were analyzed with a polarized microscope (Nikon, Melville, N.Y., http://www.nikonusa.com) to visualize collagen alignment.

Data were analyzed with a two factor ANOVA, using Tukey's post hoc test when applicable and a significance value of p<0.05. At least four samples were analyzed for biochemical assays and biomechanical tests for all groups. All data are reported as mean±standard deviation. Statistical differences between groups are denoted by a standard convention using letters. This convention illustrates significant differences between groups when the groups are not connected by the same letter. Since two experimental factors were assessed, upper and lower case letters were designated to each factor, with differentiation conditions (CM, D1, and D2) having lower case letters and self-assembly mode (EB or DC) having upper case letters.

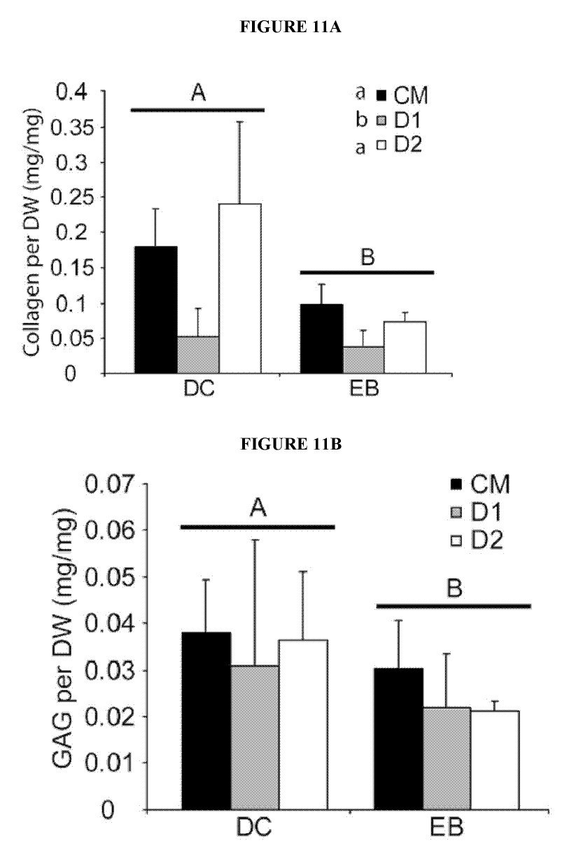

After the initial seeding of the dissociated cells (DCs) into the 3-mm agarose wells, cells coalesced within 24 hrs into constructs that were slightly smaller than the well. Over the following weeks, the spacing between cells in each construct increased as they produced ECM, causing the constructs to appear smooth and cartilaginous (FIG. 10A). The amount of EBs for each group seeded into the 5 mm wells was enough to cover the entire bottom surface initially. Over the ensuing weeks, CM and D2 constructs filled the well, while D1 constructs appeared to shrink away from the outer edges. EB constructs never achieved homogeneity during the experiment. A clear matrix connected EBs in a construct, and the constructs appeared highly hydrated (FIG. 10A).

Construct morphological measurements are shown in FIG. 10 below the gross morphological pictures. D1 constructs had significantly lower thickness and wet weight compared to CM constructs for both EB and DC groups (p<0.05), while D2 constructs were not different from either of the differentiation conditions. At t=8 wks, CM and D2 constructs demonstrated uniform staining for collagens I and II, regardless of self-assembly mode (EB or DC, FIG. 10B). D1 constructs also demonstrated uniform staining for collagen type II but no significant staining for collagen type I (FIG. 10B), for both EB and DC self-assembled constructs. Intense picrosirius red staining in all self-assembled constructs illustrated the matrix-producing capacity of the differentiated cells (FIG. 10B). Conversely, Alcian blue staining was minimal (FIG. 10B). An interesting finding with histology was that a central pocket of fluid had formed within the DC constructs (FIG. 10B). This was noted primarily in the CM and D2 constructs. At the end of the 8 wk experiment, other mesodermal tissues (bone, muscle, adipose) were not detected by histology (data not shown).

Biochemical Analysis of the Constructs.

Biochemical assays included dimethylmethylene blue (DMMB), hydroxyproline, picogreen, and ELISAs for collagens I and II. Samples were lyophilized for 48 hrs, and dry weights were measured. Previously described protocols were used for DMMB and hydroxyproline tests, and one set of samples was used for these two assays. For collagens I and II, Chondrex reagents and protocols were used (Chondrex, Redmond, Wash., http://www.chondrex.com), with the exception that constructs were digested with papain (rather than pepsin) at 4° C. for 4 days, followed by a 1 day elastase digest. The picogreen assay for DNA content was performed using this set of samples, and a multiple of 7.7 pg DNA per cell was used.

When comparing between EB and DC self-assembled groups for biochemical content, normalized by dry weight (dw), DC constructs demonstrated greater matrix production (both collagen and GAG) (p<0.05), as shown in FIG. 11. The measurements for hydroxyproline showed that the D1 DC group did not produce as much collagen (5.2% by dw) as the other two groups, with CM and D2 DC constructs producing 17.9% and 24.1% by dw, respectively (FIG. 11A). Although Alcian blue staining was not substantial, the DMMB assay demonstrated the presence of sulfated GAGs in all constructs (FIG. 11B). The water content for engineered constructs in all groups was approximately 90% (91.1±2.7% for CM DC, 85.5±5.8% for D1 DC, 89.7±5.1% for D2 DC, 92.8%±3.3% for CM EB, 94.2%2.6% for D1 EB, and 91.7±2.3% for D2 EB).

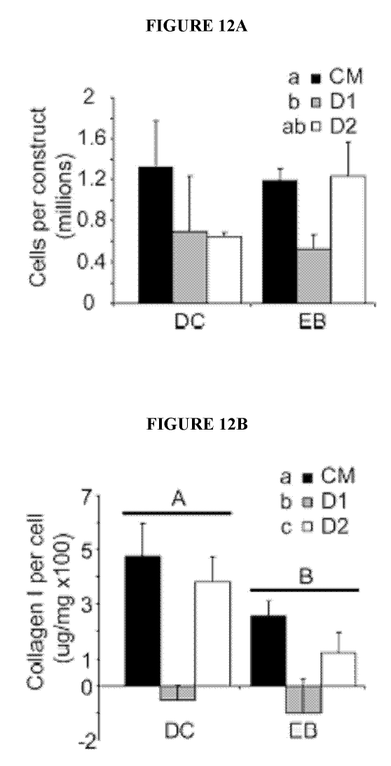

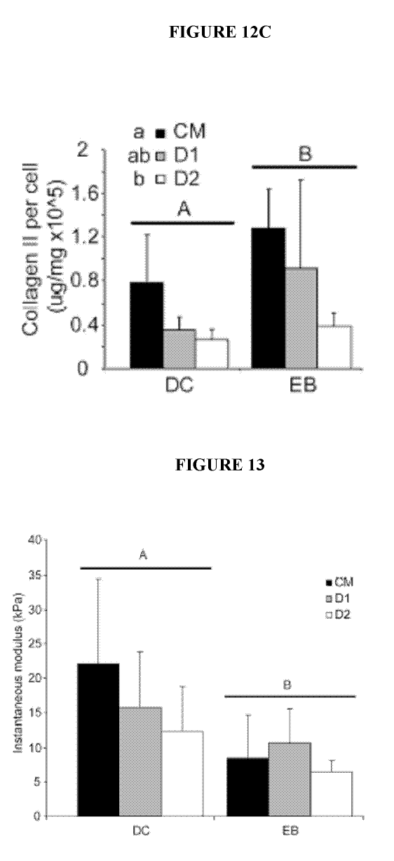

Picogreen demonstrated that the number of cells per construct was significantly different between CM and D1 groups (p<0.05), while D2 constructs were not different from the other two groups (FIG. 12A). ELISAs for collagens I and II demonstrated that the production of collagens I and II varied between each differentiation regimen and between DC and EB constructs (FIG. 12B and FIG. 12C). Specifically, collagen type I production per cell was significantly higher in CM constructs compared to the other two differentiation agents (for example, in μg×10⁻²/cell, 4.8±1.2 for CM DC, −0.5±0.5 for D1 DC, and 3.8±0.9 for D2 DC, p<0.05). D1 constructs demonstrated undetectable collagen type I, which echoed the IHC results for this group. The ELISA data also demonstrated that DC constructs had higher collagen type I and lower collagen type II production per cell than EB constructs (p<0.05). Differentiation condition was a significant factor when analyzing the collagen type II ELISA, with CM constructs having higher collagen type II content compared to D2 constructs. For example, CM DC samples had over 2-fold higher collagen type II content per cell than D2 DC samples (0.8±0.4 vs. 0.3±0.1 μg×10⁻⁵ cell, p<0.05). D1 constructs were not significantly different compared to the other two differentiation agents in terms of collagen type II content per cell.

Biomechanical Analysis of the Constructs.

Biomechanical testing included tensile testing using an Instron 5565 (Instron, Norwood, Mass., http://www.instron.us) and unconfined compression using a modified creep indentation apparatus as described in Mow V C, Gibbs M C, Lai W M, et al. Biphasic indentation of articular cartilage—II. A numerical algorithm and an experimental study. J Biomech 1989; 22:853-861.

For tensile testing, specimens were cut from the cylindrical constructs into dog-bone shapes and pulled at a strain rate of 1%/s until failure. Gauge length, thickness and width of the specimens were measured with digital calipers so that load and extension measurements could be converted to stress and strain. Similar to the whole constructs, collagen alignment of the tensile specimens was analyzed with picrosirius red staining and polarized light. For unconfined compression testing, constructs were allowed to equilibrate in PBS for 10 min, and then subjected to an instantaneous 1.96 mN test load. The creep test was allowed to run for at least 1 hr. which was long enough to achieve deformation equilibrium. With the unconfined compression creep data, intrinsic material properties of the constructs were obtained using a previously developed viscoelastic model as described in Leipzig N D and Athanasiou K A. Unconfined creep compression of chondrocytes. J Biomech 2005; 38:77-85.

Data were analyzed with a two factor ANOVA, using Tukey's post hoc test when applicable and a significance value of p<0.05. At least four samples were analyzed for biochemical assays and biomechanical tests for all groups. All data are reported as mean±standard deviation. Statistical differences between groups are denoted by a standard convention using letters. This convention illustrates significant differences between groups when the groups are not connected by the same letter. Since two experimental factors were assessed, upper and lower case letters were designated to each factor, with differentiation conditions (CM, D1, and D2) having lower case letters and self-assembly mode (EB or DC) having upper case letters.

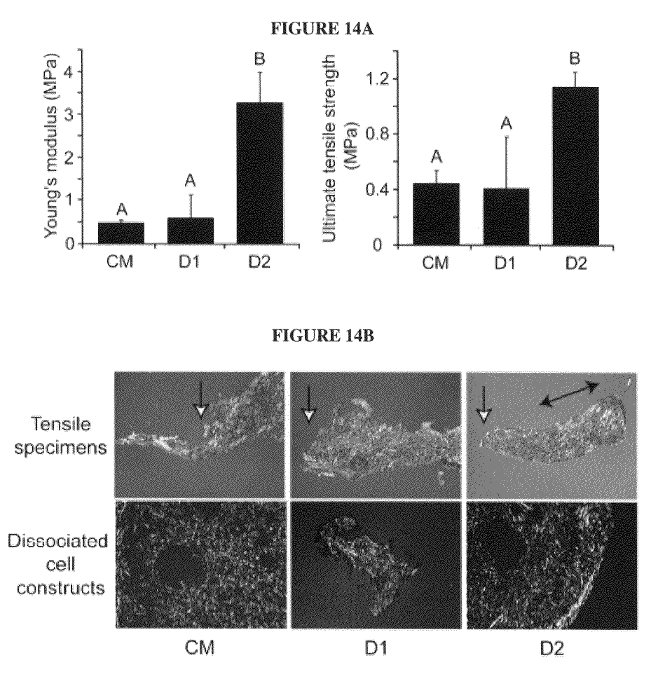

Unconfined compression testing of the self-assembled constructs demonstrated that DC constructs had a significantly higher instantaneous modulus compared to EB constructs (p<0.05), while there was no significant difference between CM, D1, and D2 constructs (FIG. 13). There was no statistical difference between any treatments in terms of their relaxed modulus (2.2±1.5 kPa for CM DC, 1.7±0.8 kPa for D1 DC, 1.3±0.3 kPa for D2 DC, 0.7±0.1 for CM EB, 1.8±0.7 kPa for D1 EB, and 0.8±0.2 kPa for D2 EB). The CM and D2 DC constructs exhibited a higher apparent viscosity than all other treatments (2778±817 kPa-s for CM DC, 1489±857 kPa-s for D1 DC, 2487±980 kPa-s for D2 DC, 539±208 kPa-s for CM EB, 1445±572 kPa-s for D1 EB, and 693±356 kPa-s for D2 EB). Tensile testing (FIG. 14 A) showed that D2 DC constructs had an over 5.5-fold higher tensile modulus (3.3±0.7 vs. 0.6±0.5 MPa) and 2.8-fold higher ultimate tensile strength compared to D1 constructs (1.1±0.1 vs. 0.4±0.3 MPa). Comparing these tensile properties of D2 to CM constructs yielded similar increases (6.6-fold and 2.8-fold, respectively). Polarized light microscopy performed directly on tensile tested specimens demonstrated collagen alignment in the direction of tensile testing for D2 DC tensile specimens while CM and D1 tensile specimens did not (FIG. 14 B). Moreover, D2 constructs exhibited a higher degree of collagen alignment than CM and D1 constructs in the untested DC samples. EB constructs were not testable under tension.

Differences were observed at t=4 wks in terms of cell morphology and at t=8 wks in terms of construct morphology (FIG. 10), biochemistry (FIG. 11 and FIG. 12), and tensile properties (FIG. 14). Since cells from each differentiation condition were cultured in the basal chondrogenic medium without exogenous growth factors during self-assembly, these data collectively indicate that the cells generated after 4 wks of EB differentiation had varying capacities to produce cartilage.

The constructs engineered according to the previous examples generally exhibited properties most similar to the fibrocartilages, particularly the TMJ disc and the outer portion of the knee meniscus. The constructs had relatively high total collagen contents (up to 24% by dw in this study vs. ˜80% by dw for native TMJ and outer meniscus), low sulfated GAG contents (about 4% by dw in this study vs. 0.6 to 10% for native TMJ and outer meniscus), and relatively high tensile properties (order of 1 MPa in this study vs. order of 10-100 MPa for the native fibrocartilages). These fibrocartilages are also notable for their high collagen type I content and low to absent collagen type II content. Both CM and D2 constructs demonstrated this pattern, while D1 constructs did not contain detectable collagen type I.

Compared to studies using biomaterials as scaffolds, as well as our original work describing self-assembly, the constructs produced by chondrogenically-differentiated hESCs have comparable collagen content (around 1 to 2% by wet weight), but lower sulfated GAG. Even though the current examples produced mostly fibrocartilage and these previous tissue-engineering studies produced hyaline-like cartilage with native chondrocytes, this comparison demonstrates the matrix-producing capacity of the differentiated hESCs. The tensile properties have been measured on the order of 1 MPa with native chondrocyte self-assembled constructs.

The most dramatic difference between differentiation conditions was revealed by the tensile testing. D2 tensile specimens exhibited the highest degree of collagen alignment, and this finding appears to account for the higher tensile modulus and ultimate tensile strength of this group (FIG. 14). Whether this is a true functional difference needs further investigation. One explanation for the apparent differences in degree of alignment and tensile properties is that the D2 cells, which had a more fibroblastic morphology (FIG. 9B), had a better ability to organize the collagen network. The link between cell shape and function has been well established in various types of cartilage. Additionally, in native cartilages, the resident cells, such as chondrocytes, remodel the matrix on a regular basis.

Another curious finding was the pocket of fluid inside of the CM and D2 constructs. Our initial self-assembly study used bovine cells and bovine serum, and encountered no fluid-filled region. A possibility for the fluid-filled interior encountered in this study is that a different cell population (chondrogenic or non-chondrogenic) accumulated in this space, but the histological evidence did not offer support of this idea.

While characterization of the differentiation process was one major goal of this study, we also determined how the differentiated hESCs responded to the transition from differentiation in EB form to tissue engineering. While constructs made with both self-assembly modes, EB and DC, expressed cartilage proteins, the gross appearance (FIG. 10), total collagen and sulfated GAG contents (FIG. 11), and biomechanical properties (compressive, FIG. 13, and tensile, FIG. 14) of the DC constructs were better. Additionally, the ELISA results (FIG. 12) suggested that the process of digesting the EBs after 4 wks of differentiation and subsequently placing the cells into agarose wells for self-assembly increases collagen type I content and decreases collagen type II content. In comparing EB and DC constructs, it is important to note that the difference in initial construct size (3 mm wells for DC constructs and 5 mm wells for EB constructs) was necessary due to difficulty with seeding the EBs into 3 mm wells. This difference in construct size between EB and DC groups necessitated comparisons normalized by cell number and dry weight. Given the marked differences found between these two groups with this analysis, it was postulated that the ECM produced by the EBs during the first 4 wks hindered cell-cell contacts and lowered the concentration of cells when they were placed in agarose molds for self-assembly. On the other hand, enzymatic dissociation of the EBs and subsequent seeding of the cells into agarose molds promoted direct cell contacts and a higher cell density. Even in normal development of cartilaginous tissues, such as articular cartilage, mesenchymal precursors aggregate at high density with direct cell contacts as an early step of chondrogenesis.

The preceding examples illustrate a new methodology to study cartilage tissue engineering with hESCs. The use of self-assembly as a tissue engineering strategy resulted in quantitative data that addressed two hypotheses. First, we investigated whether cells with different chondrogenic potentials would be generated when hESCs were exposed to distinct growth factor regimens for 4 wks. We assessed this after the cells had formed neocartilage (at t=8 wks), showing differences in the chondrogenic potential of CM (chondrogenic medium), D1 (TGF-β3 followed by TGF-β1 with IGF-I, added to CM), and D2 (TGF-β3 followed by BMP-2, added to CM) cartilage constructs in terms of morphology, biochemistry, and biomechanics. These properties also illustrated that DC constructs outperform EB constructs and thereby highlighting the importance of enzymatic dissociation of EBs prior to self-assembly. These findings represent incremental steps toward functional engineering of different types of musculoskeletal cartilages with hESCs.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

Therefore, the present invention is well adapted to attain the ends and advantages mentioned as well as those that are inherent therein. While numerous changes may be made by those skilled in the art, such changes are encompassed within the spirit of this invention as illustrated, in part, by the appended claims.

What is claimed is:

1. A method for inducing differentiation of human embryonic stem cells into chondrocytes comprising
aggregating undifferentiated human embryonic stem cells to form embryoid bodies; and
culturing the embryoid bodies, or cells dissociated from the embryoid bodies, in a culture medium, wherein the culture medium comprises a growth factor that induces chondrogenic differentiation of the embryoid bodies, or of the cells.

2. The method of claim 1 wherein culturing further comprises hypoxia.

3. The method of claim 1 wherein culturing further comprises co-culturing with somatic cells.

4. The method of claim 1 wherein culturing further comprises co-culturing with one or more somatic cells chosen from chondrocytes, fibrochondrocytes, and synoviocytes.

5. The method of claim 1 further comprising, purifying differentiated human embryonic stem cells using a density gradient method.

6. The method of claim 1 wherein the undifferentiated cells are from an embryonic stem cell bank.

7. The method of claim 1 wherein the undifferentiated cells are derived from somatic cell nuclear transfer.

8. The method of claim 1 wherein the undifferentiated cells are derived from induced pluripotent stem cells.

9. The method of claim 1 wherein the growth factor is chosen from one or more of TGF-β1, IGF-I, TGF-β3, BMP-2, and BMP-4.

10. The method of claim 1 wherein the growth factor is present in a range of from about 1 ng/mL to about 1,000 ng/mL of the culture medium.

11. The method of claim 1 wherein the culture medium is substantially free of fetal bovine serum.

12. A method of forming a scaffoldless tissue engineered construct comprising:
aggregating undifferentiated human embryonic stem cells to form embryoid bodies;
culturing the embryoid bodies, or cells dissociated from the embryoid bodies, in a culture medium, wherein the culture medium comprises a growth factor that induces chondrogenic differentiation of the embryoid bodies, or of the cells;
sedimenting the embryoid bodies, or cells dissociated from the embryoid bodies, onto a hydrogel coated culture vessel; and
allowing the sedimented embryoid bodies, or cells, to self-assemble to form a construct.

13. The method of claim 12 wherein the undifferentiated cells are from an embryonic stem cell bank.

14. The method of claim 12 wherein the undifferentiated cells are derived from somatic cell nuclear transfer.

15. The method of claim 12 wherein the undifferentiated cells are derived from induced pluripotent stem cells.

16. The method of claim 12 wherein the growth factor is chosen from one or more of TGF-β1, IGF-I, TGF-β3, BMP-2, and BMP-4.

17. The method of claim 12 wherein the growth factor is present in a range of from about 1 ng/mL to about 1,000 ng/mL of the culture medium.

18. The method of claim 12 wherein the culture medium is substantially free of fetal bovine serum.

19. The method of claim 12 further comprising, treating the construct with staurosporine or a ROCK inhibitor or both.

20. The method of claim 12 further comprising, molding the construct into a desired shape.

21. The method of claim 12 further comprising, molding the construct into a desired shape, wherein the molding comprises
transferring the construct to a shaped hydrogel negative mold,
applying a shaped hydrogel positive mold to the negative mold to form a mold-construct assembly, and
culturing the mold-construct assembly.

22. The method of claim 12 further comprising, molding the construct into a desired shape, wherein the desired shape is a shape of at least a portion of a joint of mammal, a cartilaginous tissue of a mammal, a tendon tissue of a mammal, or a ligament tissue of a mammal.

23. The method of claim 12 further comprising, molding the construct into a desired shape, wherein the desired shape is at least a portion of a femur or a temporomandibular joint.

24. The method of claim 12 further comprising, exposing the embryoid bodies, or cells dissociated from the embryoid bodies, to a pressure or a load or both.

25. A method for treating a subject comprising implanting in the subject a composition comprising at least one tissue engineered construct prepared by the method of claim 1, claim 13, or claim 17.

26. A scaffoldless tissue engineered construct prepared by the method of claim 1, claim 13, or claim 17.
